(19) United States
(12) Patent Application Publication
(10) Pub. No.: US 2025/0268556 A1
(43) Pub. Date: Aug. 28, 2025

(54) MEDICAL SYSTEM AND RECORDING MEDIA

(71) Applicant: GE Precision Healthcare LLC, Waukesha, WI (US)

(72) Inventor: Kentaro Ogata, Hino (JP)

(21) Appl. No.: 19/065,975

(22) Filed: Feb. 27, 2025

(30) Foreign Application Priority Data
Feb. 27, 2024 (JP) 2024-027949

(51) Int. Cl. A61B 6/58; A61B 6/03; A61B 6/40

(52) U.S. Cl. CPC A61B 6/585; A61B 6/032; A61B 6/4035

(57) ABSTRACT

Methods and apparatus are described herein to determine an effective voltage value V1 of a high kV on the basis of energy information of X-rays detected in a first sub-region of a detector assembly and energy information of X-rays detected in a second sub-region of the detector assembly, when the high kV is applied to an X-ray tube, determine an effective voltage value of a low kV on the basis of energy information of X-rays detected in the first sub-region of the detector assembly and energy information of X-rays detected in the second sub-region of the detector assembly, when the low kV is applied to the X-ray tube, calculate a correction coefficient required to correct for beam hardening on the basis of an effective voltage value, and calculate a correction coefficient required to correct for beam hardening on the basis of an effective voltage value.

### CROSS-REFERENCE TO RELATED APPLICATIONS

This application claims priority to Japanese Application No. 2024-027949, filed on Feb. 27, 2024, the disclosure of which is incorporated herein by reference in its entirety.

### TECHNICAL FIELD

The present invention relates to a medical system for executing beam hardening correction; and a recording medium in which an instruction for controlling the medical system is recorded.

### BACKGROUND ART

A CT system is known as a medical device that noninvasively images a subject. CT systems are widely used in hospitals and other medical facilities because they can acquire tomographic images of a subject in a short scanning time.

The CT system applies a prescribed voltage to the cathode-anode tube of an X-ray tube to generate X-rays. The generated X-rays penetrate the subject and are detected by the detector. The CT system reconstructs a CT image of the subject on the basis of data detected by the detector.

### SUMMARY OF THE INVENTION

Single energy CT (SECT) is a well-known imaging technique for CT systems. SECT is a method for obtaining a CT image of a subject by applying a prescribed voltage (e.g., 120 kV) to a cathode-anode tube of an X-ray tube to generate X-rays. However, in SECT, CT values may be close even for different substances, and identification of different substances may be difficult.

Therefore, DECT (Dual Energy CT) technology is being researched and developed. DECT is a technology that uses X-rays in different energy regions to distinguish between substances, and can acquire images that are useful for diagnosis in clinical settings, and thus is beginning to come into widespread use. In the DECT technology, a kV switching technology is known that switches the tube voltage of the X-ray tube between a low tube voltage and a high tube voltage.

Furthermore, parts of a CT system deteriorate over time as the system is used for a long period of time, and as a result, the beam hardening correction coefficient used in the CT system deviates from the ideal value. Therefore, in the CT system, calibration is periodically executed to correct the beam hardening. In particular, in calibration of DECT using k V switching, a calibration scan is executed in order to collect data corresponding to a high tube voltage and data corresponding to a low tube voltage, and a correction coefficient required for correcting beam hardening is obtained on the basis of the collected data.

On the other hand, in kV switching, it is necessary to switch between a low tube voltage and a high tube voltage in a short period of time. Ideally, when the tube voltage is switched, the voltage is preferably changed to a rectangular shape. However, in reality, when the tube voltage is switched, a certain amount of time is required for the tube voltage to reach a desired value. On the other hand, as described above, in kV switching, it is necessary to switch the voltage in a short period of time. Therefore, when the tube voltage is switched, for example, from a low tube voltage to a high tube voltage, even if the high tube voltage reaches an ideal voltage value, the tube voltage is immediately switched to the low tube voltage. Thus, the time during which the ideal voltage value is maintained is reduced. This becomes more noticeable as the kV switching speed increases. Thus, when considering high and low tube voltages in kV switching technology, the effective voltage value is considered, not the ideal voltage value of the tube voltage.

Therefore, when beam hardening correction is executed, a necessary correction coefficient is obtained on the basis of the effective voltage value of a tube voltage.

On the other hand, the effective voltage value depends on the rotation speed of a gantry and the tube current of an X-ray tube. For this reason, in calibration, it is necessary to determine in advance combinations of rotation speeds and tube currents as presets, execute a calibration scan to obtain an effective voltage value for each preset, and obtain a beam hardening correction coefficient on the basis of the effective voltage value. Therefore, as the number of presets increases, the number of calibration scans that must be executed also increases, resulting in a problem in which a great deal of time is required for calibration.

Furthermore, for a combination of rotation speed and tube current that is not preset, a calibration scan cannot be executed, and thus an effective voltage value cannot be obtained. Therefore, even if a user wishes to execute an examination of a subject using a combination of rotation speed and tube current that is not preset, an effective voltage value cannot be obtained, and thus the beam hardening correction coefficient cannot be obtained. This poses a problem in which a scan cannot be performed with a combination of rotation speed and tube current desired by the user. Thus, a technology is desired that enables a correction coefficient required for beam hardening to be easily obtained.

A first aspect of the present invention is a medical system, including an X-ray tube configured to be able to switch a tube voltage applied to the X-ray tube between a first tube voltage and a second tube voltage a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter, and one or a plurality of processors for executing the following: determining a first effective voltage value of the first tube voltage on the basis of energy information of X-rays detected in the first sub-region of the detector assembly and energy information of X-rays detected in the second sub-region of the detector assembly, when the first tube voltage is applied to the X-ray tube; determining a second effective voltage value of the second tube voltage on the basis of energy information of X-rays detected in the first sub-region of the detector assembly and energy information of X-rays detected in the second sub-region of the detector assembly, when the second tube voltage is applied to the X-ray tube; calculating a first correction coefficient required to correct for beam hardening on the basis of the first effective voltage value; and calculating a second correction coefficient required to correct for beam hardening on the basis of the second effective voltage value.

Furthermore, a second aspect of the present invention is a non-transitory storing medium in which is stored an instruction executed by one or a plurality of processors of a medical system including an X-ray tube configured to be able to switch a tube voltage applied to the X-ray tube between a first tube voltage and a second tube voltage, a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter, and the one or plurality of processors, wherein the instruction, upon being executed by the one or plurality of processors, causes the one or plurality of processors to execute the following: determining a first effective voltage value of the first tube voltage on the basis of energy information of X-rays detected in the first sub-region of the detector assembly and energy information of X-rays detected in the second sub-region of the detector assembly, when the first tube voltage is applied to the X-ray tube; determining a second effective voltage value of the second tube voltage on the basis of energy information of X-rays detected in the first sub-region of the detector assembly and energy information of X-rays detected in the second sub-region of the detector assembly, when the second tube voltage is applied to the X-ray tube; calculating a first correction coefficient required to correct for beam hardening on the basis of the first effective voltage value; and calculating a second correction coefficient required to correct for beam hardening on the basis of the second effective voltage value.

In the present invention, X-rays are irradiated from an X-ray tube at a first tube voltage, and a detector assembly detects X-rays that have passed through a filter and X-rays that have not passed through the filter, thereby obtaining a first effective voltage value of the first tube voltage. Furthermore, X-rays are irradiated from the X-ray tube at a second tube voltage, and the detector assembly detects X-rays that have passed through the filter and X-rays that have not passed through the filter, thereby obtaining a second effective voltage value of the second tube voltage. Therefore, even if a combination of operating conditions of the medical system (e.g., the rotation speed of a gantry and the tube current of an X-ray tube) is not changed during calibration, effective voltage values of the first tube voltage and the second tube voltage can be obtained and a correction coefficient corresponding to each effective voltage value can be calculated. This makes it possible to reduce the time required for calibration, and to execute a scan of a subject under a combination of operating conditions desired by a user.

### BRIEF DESCRIPTION OF THE DRAWINGS

FIG. 1 is a block diagram of a CT system 100 of the present embodiment;

FIG. 2 is an explanatory diagram of a detector assembly 108;

FIG. 3 is an enlarged view of a reference region 109 of the detector assembly 108;

FIG. 4 is a flowchart of a method for creating a baseline;

FIG. 5 is an explanatory diagram of a method for calculating an index value M;

FIG. 6 is an explanatory diagram of a method for creating a baseline;

FIG. 7 is a diagram depicting a baseline 60;

FIG. 8 is a diagram depicting calibration data 70 obtained by a calibration scan;

FIG. 9 is a flowchart for generating an image of a subject 112;

FIG. 10 is an explanatory diagram of a method for setting a view number;

FIG. 11 is an explanatory diagram of a method for calculating a first index value M (=MV1) corresponding to a view v1 (high kV);

FIG. 12 is a diagram depicting a first effective voltage value V1 corresponding to the first index value MV1;

FIG. 13 is an explanatory diagram of a method for determining correction coefficient data;

FIG. 14 is an explanatory diagram of a method for calculating a second index value M(=MV2) corresponding to a view v2 (low kV);

FIG. 15 depicts a second effective voltage value V2 corresponding to the second index value MV2;

FIG. 16 is an explanatory diagram of a method for determining correction coefficient data; and

FIG. 17 is a diagram depicting an example of the relationship between the effective voltage value and the view when kV switching is executed.

### BRIEF DESCRIPTION OF THE DRAWINGS

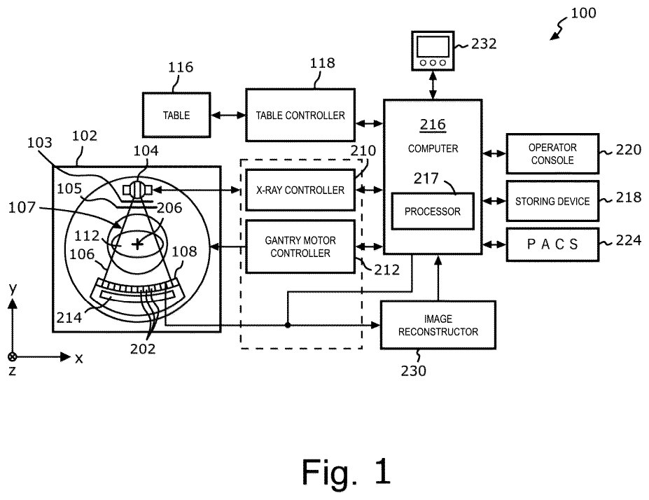

An embodiment for carrying out the invention will be described below, but the present invention is not limited to the following embodiment. FIG. 1 is a block diagram of a CT system 100 of the present embodiment. The CT system 100 includes a gantry 102 and a table 116. The gantry 102 includes a bore 107 and a subject 112 is transported through the bore 107 to scan the subject 112. An X-ray tube 104, a filter part 103, a pre-collimator 105, a detector assembly 108, and the like are attached to the gantry 102.

The X-ray tube 104 generates X-rays when a prescribed voltage is applied to the cathode-anode tube. The X-ray tube 104 is configured to be rotatable on a path centered on a rotation axis 206 within the XY plane. Herein, the Z direction represents the body-axis direction, the Y direction represents the vertical direction (the height direction of the table 116), and the X direction represents the direction perpendicular to the Z and Y directions. In the present embodiment, the X-ray tube 104 supports a rapid kV switching scheme in which the tube voltage applied to the X-ray tube 104 can be switched between a first tube voltage and a second tube voltage. Note that in the present embodiment, the CT system 100 includes one X-ray tube 104, but may include two X-ray tubes 104.

The filter part 103 includes, for example, a flat plate filter and/or a bow-tie filter. The pre-collimator 105 is a member that narrows the X-ray irradiation range such that X-rays are not irradiated in unwanted regions. The detector assembly 108 includes a plurality of detector elements 202. The plurality of detector elements 202 detect an X-ray beam 106 that is irradiated from the X-ray tube 104 and passes through the subject 112, such as a patient or the like. Therefore, the X-ray detector assembly 108 can acquire projection data for each view.

The projection data detected by the detector assembly 108 is collected by a DAS 214. The DAS 214 executes a prescribed processing, including sampling, digital conversion, and the like, on the collected projection data. The processed projection data is transmitted to a computer 216. The computer 216 stores the data from the DAS 214 in a storing device 218. The storing device 218 includes one or more recording medium that stores a program, an instruction to be executed by a processor, and the like. The recording medium may be, for example, one or more non-transitory computer-readable recording medium. The storing device 218 may include, for example, hard disk drives, floppy disk drives, compact disc-rewriteable (CD-R/W) drives, digital versatile disk (DVD) drives, flash drives, and/or solid state recording drives.

The computer 216 includes one or a plurality of processors 217. The computer 216 uses one or a plurality of processors to output commands and parameters to the DAS 214, X-ray controller 210, and/or gantry motor controller 212, to control system operations such as data acquisition and/or processing. Furthermore, the computer 216 uses one or a plurality of processors to execute various processes such as signal processing, data processing, image processing, and the like in each step of the flow described later. Note that in FIG. 1, one or a plurality of the processors 217 are included in the computer 216, but one or a plurality of the processors 217 may be provided so as to be distributed between the computer 216 and another component (e.g., X-ray controller 210, gantry motor controller 212, table motor controller 118, or the like).

An operator console 220 is linked to the computer 216. An operator can enter prescribed operator inputs related to the operation of the CT system 100 into the computer 216 by operating the operator console 220. The computer 216 receives operator input, including commands and/or scan parameters, via the operator console 220 and controls system operation on the basis of that operator input. The operator console 220 can include a keyboard (not depicted) or touch screen for the operator to specify commands and/or scan parameters.

The X-ray controller 210 controls the X-ray tube 104 on the basis of an instruction from the computer 216. Furthermore, the gantry motor controller 212 controls a gantry motor to rotate a component, such as the X-ray tube 104, detector assembly 108, or the like, on the basis of an instruction from the computer 216.

FIG. 1 depicts only one operator console 220, but two or more operator consoles may be linked to the computer 216. Furthermore, the CT system 100 may also allow a plurality of remotely located displays, printers, workstations, and/or similar devices to be linked via, for example, wired and/or wireless networks. In one embodiment, for example, the CT system 100 may include or be linked to a Picture Archiving and Communication System (PACS) 224. In a typical implementation, a PACS 224 may be linked to a remote system such as a radiology department information system, hospital information system, and/or internal or external network (not depicted) or the like.

The computer 216 provides instructions to a table motor controller 118 to control the table 116. The table motor controller 118 can control the table motor so as to move the table 116 on the basis of the instructions received. For example, the table motor controller 118 can move the table 116 such that the subject 112 is positioned appropriately for imaging.

As mentioned above, the DAS 214 samples and digitally converts the projection data acquired by the detector elements 202. The image reconstructor 230 then reconstructs the image using the sampled and digitally converted data. The image reconstructor 230 includes one or a plurality of processors, which can execute image reconstruction processing. In FIG. 1, the image reconstructor 230 is depicted as a separate component from the computer 216, but the image reconstructor 230 may form a part of the computer 216. Furthermore, the computer 216 may also perform one or a plurality of functions of the image reconstructor 230. Furthermore, the image reconstructor 230 may be located away from the CT system 100 and operatively connected to the CT system 100 using a wired or wireless network.

The image reconstructor 230 can store the reconstructed image in the storing device 218. The image reconstructor 230 may also transmit the reconstructed image to the computer 216. The computer 216 can transmit the reconstructed image and/or patient information to a display device 232 communicatively linked to the computer 216 and/or image reconstructor 230.

The various methods and processes described in the present specification can be recorded as executable instructions on a non-transitory recording medium within the CT system 100. The executable instructions may be stored on a single recording medium or distributed across a plurality of recording media. One or more processors provided in the CT system 100 execute the various methods, steps, and processes described in the present specifications in accordance with the instructions recorded on a recording medium.

The CT system 100 is configured as described above. In the present embodiment, the CT system is configured to be able to image a subject using kV switching technology, which switches the tube voltage of the X-ray tube 104 between a high tube voltage (hereinafter referred to as “high kV”) and a low tube voltage (hereinafter referred to as “low kV”).

On the other hand, X-rays used in the CT system are polychromatic X-rays having a wide energy distribution from low energy to high energy, and are not monochromatic X-rays having a single energy level. This causes a problem of producing beam hardening artifacts. Thus, in the CT system, a CT image is reconstructed on the basis of a correction coefficient required for beam hardening correction.

However, an optimum value of the correction coefficient depends on the usage state of the CT system, and therefore, the correction coefficient needs to be periodically corrected. Thus, a user of the CT system periodically executes calibration. In particular, in calibration of DECT using kV switching, kV switching is performed to alternately switch the tube voltage between high kV and low kV, X-rays are detected by the detector assembly, and a correction coefficient required to correct beam hardening is obtained on the basis of the X-ray data detected by the detector assembly.

On the other hand, in kV switching, it is necessary to switch between low kV and high kV in a short period of time. Ideally, when the tube voltage is switched, the voltage is preferably changed to a rectangular shape. However, in reality, when the tube voltage is switched, a certain amount of time is required for the tube voltage to reach a desired value. On the other hand, as described above, in kV switching, it is necessary to switch the voltage in a short period of time. Therefore, when the tube voltage is switched, for example, from a low kV to a high kV, even if the high kV reaches an ideal voltage value, the tube voltage is immediately switched to the low kV. Thus, the time during which the ideal voltage value is maintained is reduced. This becomes more noticeable as the kV switching speed increases. Thus, when considering high kV and low kV in kV switching technology, the effective voltage value is considered, not the ideal voltage value of the tube voltage. Therefore, when beam hardening correction is executed, a necessary correction coefficient is obtained on the basis of the effective voltage value.

On the other hand, the effective voltage value depends on the rotation speed of a gantry and the tube current of the X-ray tube 104. For this reason, in calibration, it is necessary to prepare in advance combinations of rotation speeds and tube currents as presets, and execute a calibration scan to obtain an effective voltage value for each preset, and beam hardening correction must be performed on the basis of the effective voltage value. For example, when considering four rotation speeds r1, r2, r3, and r4 and tube currents A1, A2, A3, and A4, the following 16 combinations of rotation speed and tube current must be determined as presets and a calibration scan must be executed for the 16 presets.
(r1, A1), (r1, A2), (r1, A3), (r1, A4)
(r2, A1), (r2, A2), (r2, A3), (r2, A4)
(r3, A1), (r3, A2), (r3, A3), (r3, A4)
(r4, A1), (r4, A2), (r4, A3), (r4, A4)

Therefore, as the number of presets increases, the number of calibration scans that must be executed also increases, resulting in a problem in which a great deal of time is required for calibration.

Furthermore, the effective voltage value generally differs for each preset. Therefore, the effective voltage values obtained for the abovementioned 16 combinations cannot be used for combinations other than the abovementioned 16 combinations. Therefore, if the combination of rotation speed and tube current desired by the user is not included in the abovementioned 16 combinations, there is a problem that a scan cannot be executed under a desired condition of the user.

Therefore, in order to address the abovementioned problem, the inventors have conducted extensive research and devised a method for calculating the effective voltage value in a simple manner. A method for calculating the effective voltage value in the present embodiment will be described below.

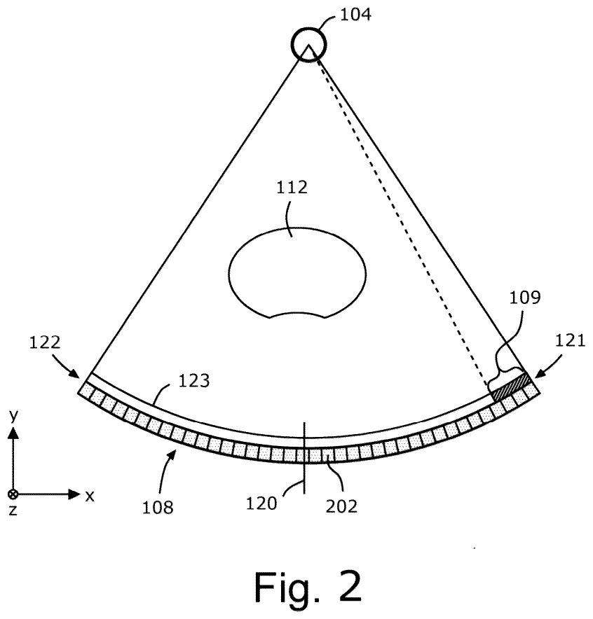

FIGS. 2 to 7 are explanatory diagrams of a method for calculating the effective voltage value in the present embodiment. FIG. 2 is an explanatory diagram of the detector assembly 108. The detector assembly 108 includes a plurality of the detector elements 202. The detector assembly 108 is provided with a reference region 109. Energy information of X-rays detected in the reference region 109 is used to calculate an index value M, which will be described later. The index value M will be described in detail later. The reference region 109 is provided at a position as far away as possible from a center position 120 in the X direction of the detector assembly 108 such that, during scanning of the subject 112, X-rays irradiated from the X-ray tube 104 that do not pass through the subject 112 can be detected. In the present embodiment, the reference region 109 is provided at one end part 121 of the detector assembly 108. Note that the reference region 109 may be provided not only at the one end part 121 of the detector assembly 108 but also at an opposite end part 122 of the detector assembly 108. In the present embodiment, the description will be continued with the reference region 109 being provided only at the one end part 121 of the detector assembly 108.

Furthermore, the detector assembly 108 includes a filter 123 to reduce image quality deterioration caused by scattered radiation. The detector collimator 123 is disposed on the X-ray tube 104 side with respect to the detector elements 202.

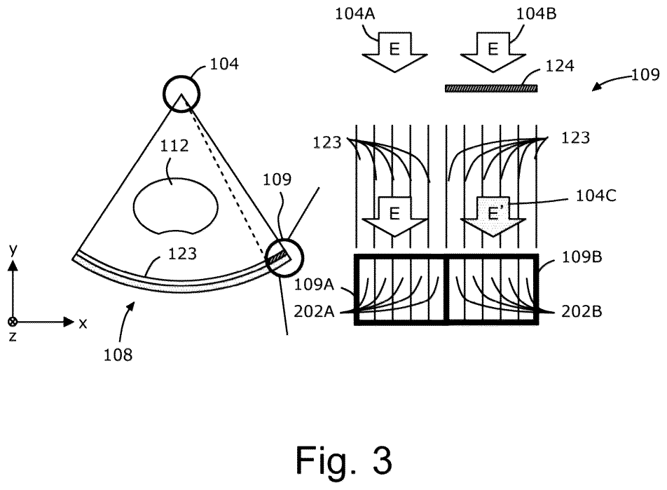

FIG. 3 is an enlarged view of the reference region 109 of the detector assembly 108. The detector collimator 123 is provided on an extension of a boundary between adjacent detector elements 202. Furthermore, FIG. 3 depicts X-rays 104A and 104B. The X-rays 104A represent X-rays advancing toward a detector element 202A of the plurality of detector elements 202 disposed in the reference region 109 of the detector assembly 108. Furthermore, the X-rays 104B represent X-rays advancing toward a detector element 202B of the plurality of detector elements 202 disposed in the reference region 109 of the detector assembly 108.

The detector assembly 108 includes a filter 124. The filter 124 may be manufactured using, for example, copper, molybdenum, tungsten, or the like. The filter 124 is not disposed in the path of the X-rays 104A, but is disposed in the path of the X-rays 104B. The X-rays 104A are detected by the detector element 202A without passing through filter 124. On the other hand, the X-rays 104B pass through the filter 124, and X-rays 104C, the energy of which has been absorbed by the filter 124, are detected by the detector element 202B. Therefore, the reference region 109 of the detector assembly 108 has a first sub-region 109B in which X-rays that have passed through the filter 124 are detected and a second sub-region 109A in which X-rays that have not passed through the filter 124 are detected.

In the present embodiment, as described above, the CT system 100 includes the detector assembly 108 having the filter 124 in the reference region 109, and the effective voltage value of the X-ray tube 104 can be obtained on the basis of the X-rays detected in the reference region 109 of the detector assembly 108. However, in order to obtain the effective voltage value, a baseline that is used to obtain the effective voltage value must be prepared in advance. The baseline can generally be created when installation work is performed to install the CT system 100 in a facility such as a hospital or the like, or when replacement work of the X-ray tube is performed. A method for creating a baseline will be described below.

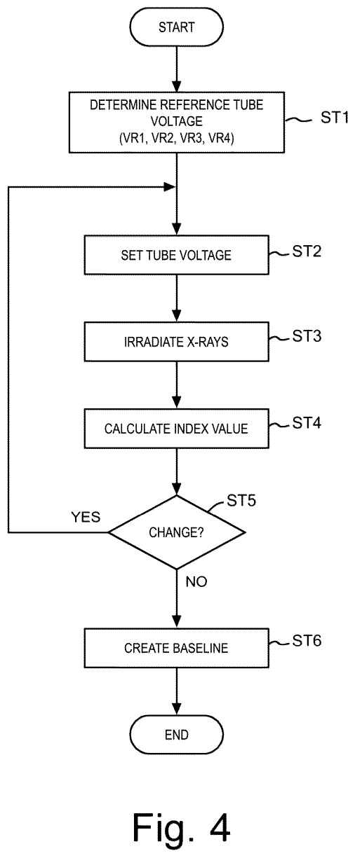

FIG. 4 is a flowchart of the method for creating a baseline. In step ST1, a reference tube voltage (hereinafter referred to as “reference tube voltage”) for a tube voltage used to create a baseline is determined. In the present embodiment, four reference tube voltages VR1, VR2, VR3, and VR4 are determined as reference tube voltages. The reference tube voltages VR1 to VR4 can be determined on the basis of tube voltages actually used in kV switching. In the present embodiment, the reference tube voltages are VR1=80 kV, VR2=100 kV, VR3=120 kV, and VR4=140 kV, but other reference tube voltages may be adopted.

Note that in the present embodiment, the four reference tube voltages VR1 to VR4 are determined as the reference tube voltages used to create the baseline. However, five or more reference tube voltages may be determined as the reference tube voltages used to create the baseline, or three or less reference tube voltages may be determined as the reference tube voltages used to create the baseline. Note that if the number of reference tube voltages is too small, the reliability of the accuracy of the effective tube voltage may decrease. On the other hand, if the number of reference tube voltages is too large, a great deal of time is required for data collection. Thus, the number of reference tube voltages is preferably determined in consideration of a balance thereof. As an example, the four reference tube voltages VR1 to VR4 employed in the present embodiment are preferably determined as the reference tube voltages used to create the baseline. After the four reference tube voltages VR1 to VR4 have been determined, the process proceeds to step ST2.

In step ST2, the tube voltage of the X-ray tube 104 is set to a reference tube voltage used to create the baseline. Herein, of the four reference tube voltages VR1 to VR4 determined in step ST1, the reference tube voltage VR1 (=80 kV) is first selected, and the tube voltage of the X-ray tube 104 is set to the reference tube voltage VR1 (=80 kV). Once the reference tube voltage is set, the process proceeds to step ST3.

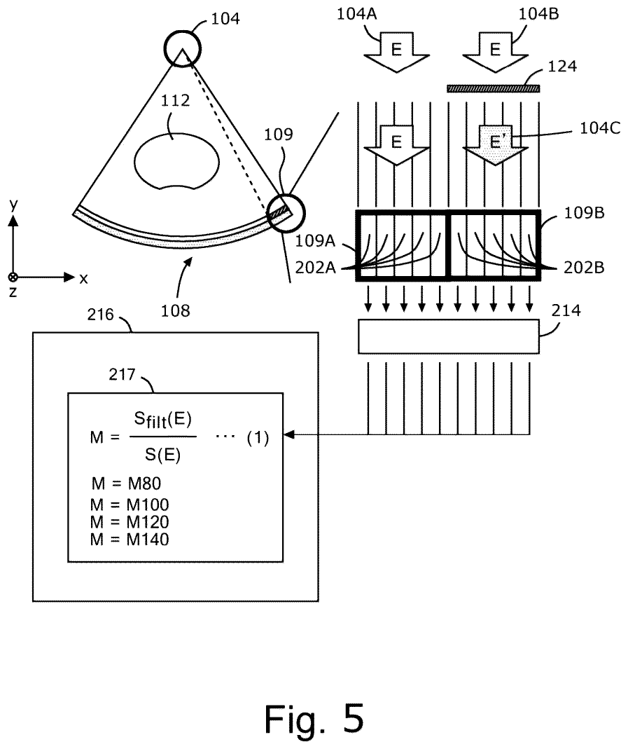

In step ST3, the reference tube voltage VR1 (=80 kV) is applied to the X-ray tube 104, and X-rays are irradiated from the X-ray tube 104. The X-rays irradiated from the X-ray tube 104 are detected by the detector assembly 108. The X-rays detected by the detector assembly 108 are output as analog data to the DAS 214 as depicted in FIG. 5. The DAS 214 samples the analog data received from detector assembly 108 and converts the analog data to digital data for subsequent processing. The digital data is output to the computer 216.

In step ST4, the computer processor 217 calculates, on the basis of the digital data, an index value M representing the difference between energy information of X-rays detected in the first sub-region 109B of the reference region 109 (i.e., X-rays that have passed through the filter 124) and energy information of X-rays detected in the second sub-region 109A of the reference region 109 (i.e., X-rays that have not passed through the filter 124). A method for calculating the index value M will be described with reference to FIG. 5.

In the present embodiment, the index value M can be calculated by the following equation:

$$M = S f i l t ( E ) / S ( E ) \qquad ( 1 )$$

Herein, M: index value
Sfilt(E): Signal value including energy information of X-rays detected by the detector element 202B of the first sub-region 109B (i.e., X-rays that have passed through the filter 124)
S(E): Signal value including energy information of X-rays detected by the detector element 202A of the second sub-region 109A (i.e., X-rays that have not passed through the filter 124)

Therefore, the index value M can be expressed as a ratio between the signal value Sfilt(E) including energy information of X-rays that have passed through the filter 124 and been detected in the first sub-region 109B and the signal value S(E) including energy information of X-rays that have not passed through the filter 124 and have been detected in the second sub-region 109A.

Note that the signal value Sfilt(E) can be obtained on the basis of a signal obtained from one or more detector element 202B of the five detector elements 202B. For example, the signal value Sfilt(E) may be obtained on the basis of a representative value (maximum value, integral value, or the like) of a signal obtained from one detector element 202B of the five detector elements 202B, or the like, or obtained on the basis of the average value and/or total value of representative values (maximum values, integral values, or the like) of signals obtained from two or more detector elements 202B of the five detector elements 202B, or the like.

Similarly, the signal value S(E) can be obtained on the basis of a signal obtained from one or more detector element 202A of the five detector elements 202A. For example, the signal value S(E) may be obtained on the basis of a representative value (maximum value, integral value, or the like) of a signal obtained from one detector element 202A of the five detector elements 202A, or the like, or obtained on the basis of the average value and/or total value of representative values (maximum values, integral values, or the like) of signals obtained from two or more detector elements 202A of the five detector elements 202A, or the like.

Therefore, by setting the tube voltage of the X-ray tube 104 to 80 kV and obtaining the signal values Sfilt(E) and S(E) on the basis of the energy of X-rays detected in the reference region 109 of the detector assembly 108, the index value M (=M80) corresponding to the reference tube voltage VR1 (=80 kV) can be calculated from equation (1).

As described above, when calculating the index value M80, the tube voltage of the X-ray tube 104 is constant at 80 kV. Therefore, there is no need to switch the tube voltage between high and low kVs while the gantry is rotating, and thus the value of the tube voltage remains stable while the gantry is rotating. For this reason, the index value M80 can be calculated without being affected by the rotation speed of the gantry. Furthermore, the index value M80 is a value calculated on the basis of the X-ray energy information. The energy of the X-rays basically depends on the tube voltage of the X-ray tube 104 and does not depend so much on the tube current, and therefore, the index value M80 can be calculated without being affected by the tube current.

Thus, the index value M80 corresponding to the reference tube voltage VR1 (=80 kV) can be calculated without being substantially affected by the rotation speed of the gantry or the tube current. After calculating the index value M80, the index value M80 is stored in the storing device, and the process proceeds to step ST5.

In step ST5, whether or not to change the reference tube voltage is determined. Herein, of the four reference tube voltages VR1 to VR4, only the index value M80 of the reference tube voltage VR1 is calculated. Therefore, the process returns to step ST2 to calculate the index values M of the remaining reference tube voltages.

In step ST2, the reference tube voltage of the X-ray tube 104 is set to another voltage value. Herein, of the four reference tube voltages VR1 to VR4 determined in step ST1, the reference tube voltage VR2 (=100 kV) is selected, and the tube voltage of the X-ray tube 104 is set to the reference tube voltage VR2 (=100 kV).

In step ST3, the reference tube voltage VR2 (=100 kV) is applied to the X-ray tube 104, and X-rays are irradiated from the X-ray tube 104. The X-rays irradiated from the X-ray tube 104 are detected by the detector assembly 108. The X-rays detected by the detector assembly 108 are output as analog data to the DAS 214 as depicted in FIG. 5. The DAS 214 samples the analog data received from detector assembly 108 and converts the analog data to digital data for subsequent processing. The digital data is output to the computer 216.

In step ST4, the processor 217 calculates an index value M100 corresponding to the reference tube voltage VR2 using equation (1), and stores the index value M100 in the storing device. Furthermore, the process proceeds to step ST5, and whether or not to change the reference tube voltage is determined. Herein, of the four reference tube voltages VR1 to VR4, the index values M80 and M100 of the two reference tube voltages VR1 and VR2 have been calculated, but the index values for the remaining two reference tube voltages VR3 and VR4 have not yet been calculated. Therefore, the process returns to step ST2 to calculate the index values of the remaining reference tube voltages.

Similarly, the loop of steps ST2 to ST5 is repeatedly executed until an index value M120 corresponding to the reference tube voltage VR3 (=120 kV) and an index value M140 corresponding to the reference tube voltage VR4 are calculated. Furthermore, once the index value M140 corresponding to the reference tube voltage VR4 has been calculated, the process proceeds to step ST5.

In step ST5, whether or not to change the reference tube voltage is determined. Herein, the index values M80 to M140 corresponding to all of the four reference tube voltages VR1 to VR4 are calculated. Therefore, the process proceeds to step ST6.

In step ST6, a baseline used to determine the effective voltage value is created on the basis of the four calculated index values M80 to M140.

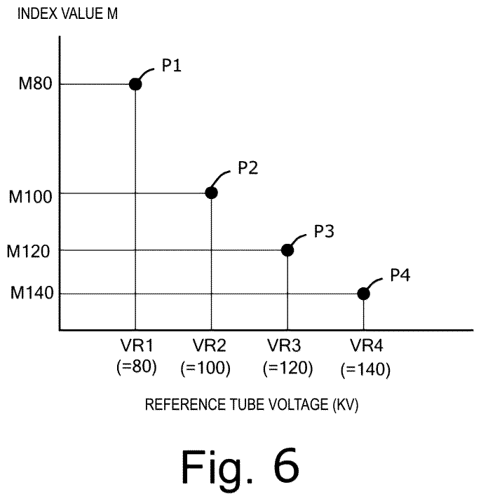

FIG. 6 is an explanatory diagram of a method for creating a baseline. The computer processor 217 associates the reference tube voltages VR1 to VR4 with the index values M80 to M140 on a two-dimensional coordinate system.

Point P1 is a point that associates the reference tube voltage VR1 with the index value M80 calculated for the reference tube voltage VR1. Point P2 is a point that associates the reference tube voltage VR2 with the index value M100 calculated for the reference tube voltage VR2. Point P3 is a point that associates the reference tube voltage VR3 with the index value M120 calculated for the reference tube voltage VR3. Point P4 is a point that associates the reference tube voltage VR4 with the index value M140 calculated for the reference tube voltage VR4.

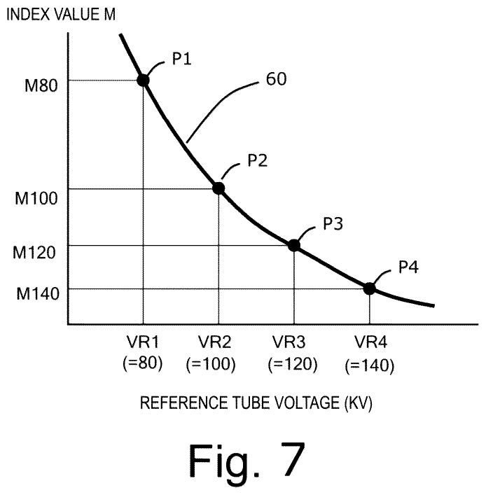

Furthermore, the processor 217 creates a baseline representing the relationship between the tube voltage and the index value M on the basis of the points P1 to P4. The baseline can be obtained, for example, as a curve that optimally fits points P1 to P4. FIG. 7 depicts a baseline 60 obtained thereby. Data representing the baseline 60 may be stored in a storing device of the CT system 100 or in an external storing device capable of communicating with the CT system 100.

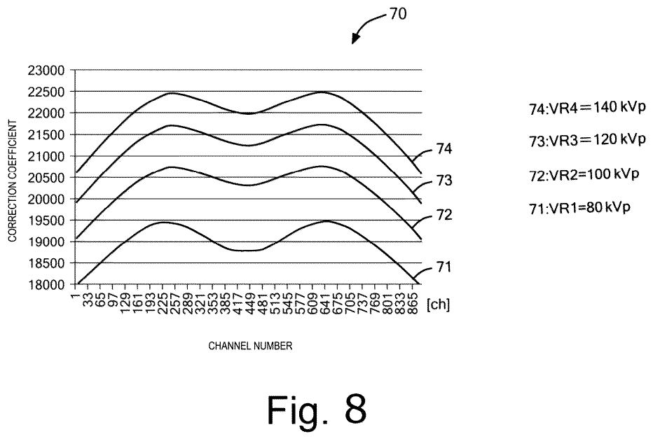

After the baseline 60 is determined, a calibration scan is executed for each of the abovementioned reference tube voltages VR1 to VR4 to acquire calibration data required for correcting beam hardening. FIG. 8 is a diagram depicting calibration data 70 obtained by the calibration scan.

The calibration data 70 includes a plurality of reference correction coefficient data 71 to 74. The horizontal axis of the calibration data 70 represents the channel number of the detector assembly 108, and the vertical axis represents the correction coefficient.

The reference correction coefficient data 71 corresponds to the reference tube voltage VR1 (=80 kV) and includes correction coefficients obtained for each channel of the detector assembly 108. The reference correction coefficient data 72 corresponds to the reference tube voltage VR2 (=100 kV) and includes correction coefficients obtained for each channel of the detector assembly 108. The reference correction coefficient data 73 corresponds to the reference tube voltage VR3 (=120 kV) and includes correction coefficients obtained for each channel of the detector assembly 108. The reference correction coefficient data 74 corresponds to the reference tube voltage VR4 (=140 kV) and includes correction coefficients obtained for each channel of the detector assembly 108.

The calibration data 70 including these reference correction coefficient data 71 to 74 is stored in a storing device of the CT system 100 or an external storing device capable of communicating with the CT system 100.

Thereby, during installation work of the CT system 100, the baseline 60 and the reference correction coefficient data 71 to 74 corresponding to the reference tube voltages VR1 to VR4 can be obtained.

Next, an example of a method for acquiring an image of the subject 112 using the abovementioned baseline 60 and calibration data 70 will be described.

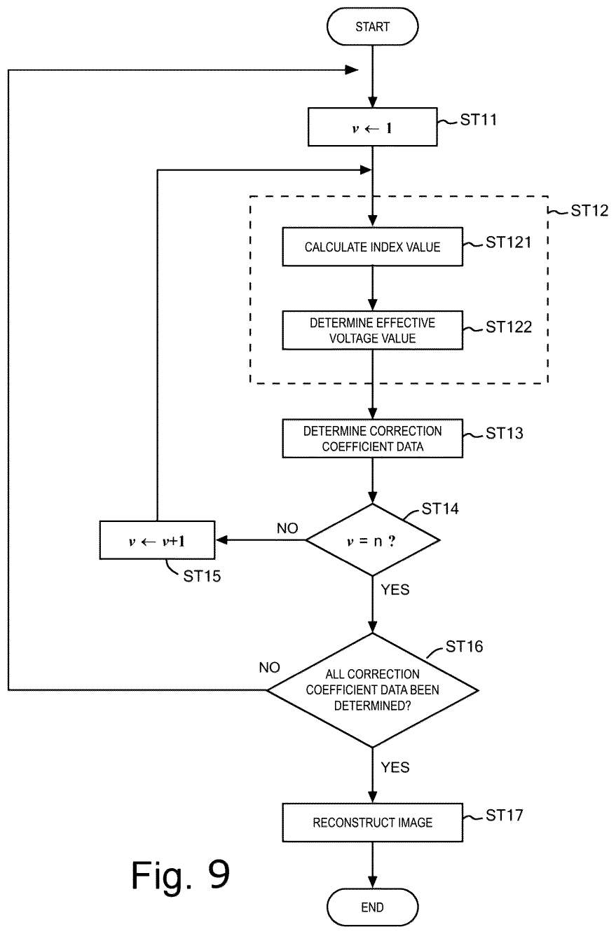

First, a scan of the subject 112 is executed using the CT system 100. The detector assembly 108 detects the X-rays, which are then output to the DAS 214. The DAS 214 samples the analog data received from detector assembly 108 and converts the analog data to digital data for subsequent processing. The digital data is output to the computer 216. The computer processor 217 generates an image of the subject 112 on the basis of the digital data (see FIG. 9).

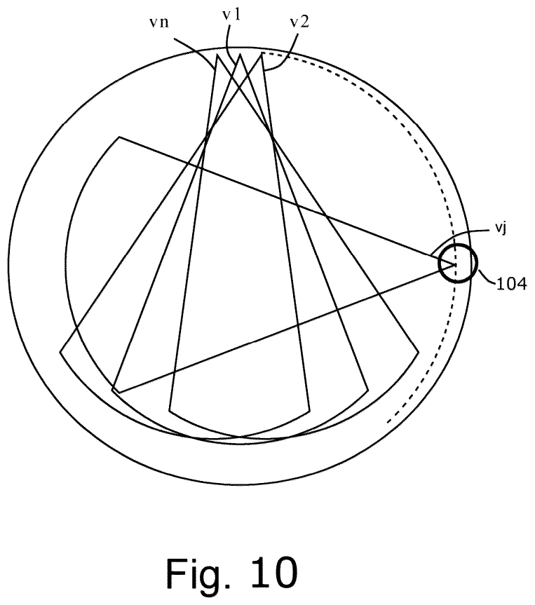

FIG. 9 is a flowchart for generating an image of the subject 112. In step ST11, the processor 217 sets the view number of a view for which a correction coefficient is to be obtained. FIG. 10 is an explanatory diagram of a method for setting the view number. In the present embodiment, projection data of n views v1 to vn are acquired for one rotation of the X-ray tube. The processor 217 identifies a view for which a correction coefficient is to be obtained from among views v1 to vn, and sets the view number of the identified view as the view number of the view for which a correction coefficient is to be obtained. Herein, a view number v=1 of the view v1 is set as the view number of the view for which the correction coefficient is to be obtained. Therefore, the processor 217 sets v=1. After the view number is set, the process proceeds to step ST12.

In step ST12, the processor 217 determines an effective voltage value for each view on the basis of the signal value including energy information of X-rays detected in the first sub-region 109B and the signal value including energy information of X-rays detected in the second sub-region 109A. Herein, v=1 is set, and therefore, a case shall be considered where an effective voltage value corresponding to view v1 of view number v=1 is obtained. The view v1 may be a view corresponding to a high kV or a view corresponding to a low kV. Herein, the view v1 is a view corresponding to high kV.

Note that step ST12 includes steps ST121 and ST122, and therefore, steps ST121 and ST122 will be described in order.

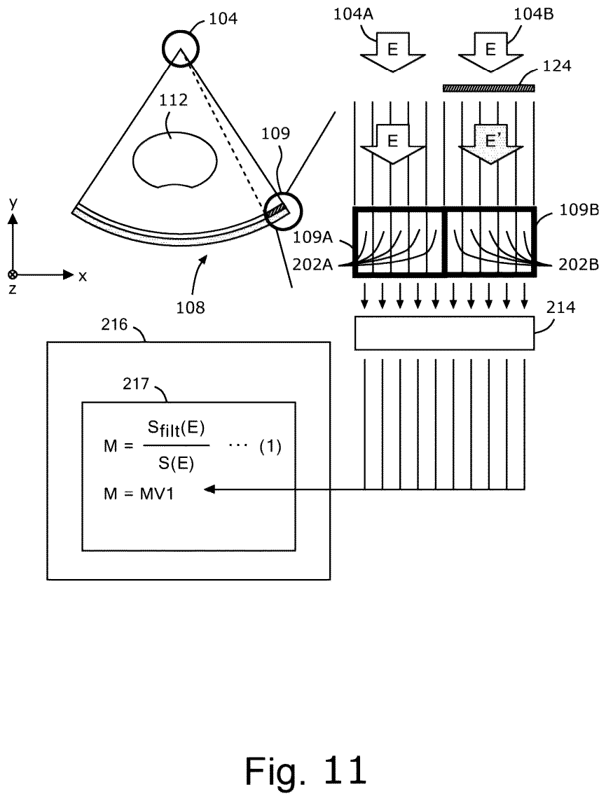

In step ST121, the processor 217 calculates a first index value M (=MV1) corresponding to the view v1 (high kV) by using equation (1) (see FIG. 11).

FIG. 11 is an explanatory diagram of a method for calculating the first index value M (=MV1) corresponding to the view v1 (high kV). The processor 217 determines the signal value Sfilt(E) and the signal value S(E) in equation (1) on the basis of data obtained from the reference region 109. The signal value Sfilt(E) is a first signal value including energy information of X-rays detected in the first sub-region 109B, and the signal value S(E) is a second signal value including energy information of X-rays detected in the second sub-region 109A. The processor substitutes the signal value Sfilt(E) and the signal value S(E) into equation (1) to calculate the first index value M (=MV1) corresponding to the view v1 (high kV). Once the first index value MV1 has been calculated, the process proceeds to step ST122.

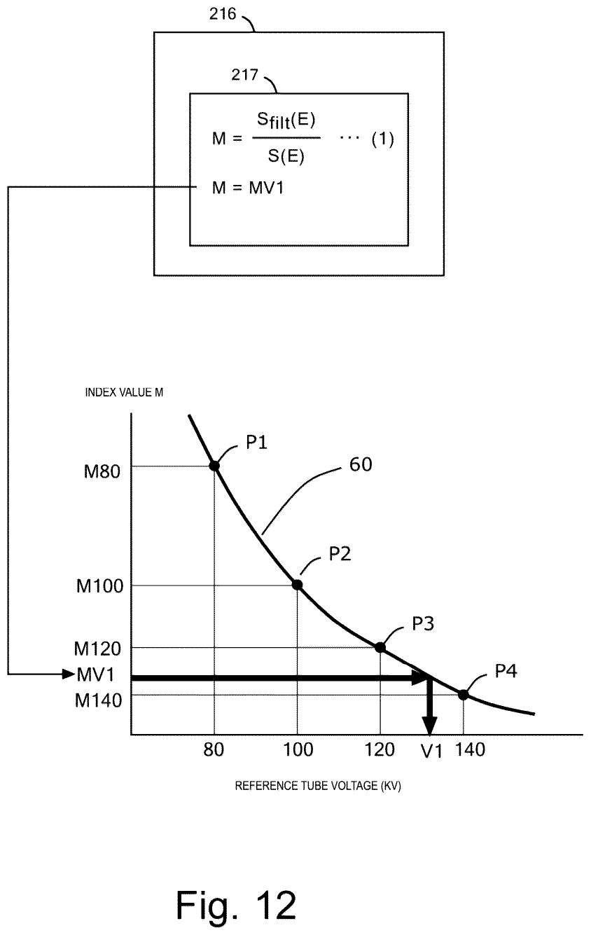

In step ST122, the processor 217 determines a first effective voltage value corresponding to the first index value MV1. FIG. 12 depicts the first effective voltage value V1 corresponding to the first index value MV1. The processor 217 reads the baseline 60 from the storing device, and identifies the first effective voltage value V1 corresponding to the first index value MV1 on the basis of the baseline 60. V1 is, for example, V1=132 kV. After the effective voltage value is determined, the process proceeds to step ST13.

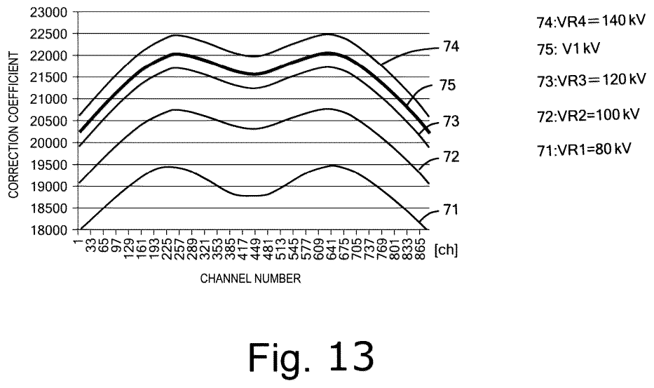

In step ST13, the processor 217 calculates correction coefficient data corresponding to the high kV view v1 on the basis of the first effective voltage value V1 determined in step ST12 (see FIG. 13).

FIG. 13 is an explanatory diagram of a method for determining the correction coefficient data. The processor 217 interpolates correction coefficient data 75 corresponding to the first effective voltage value V1 on the basis of the first effective voltage value V1 and the plurality of reference correction coefficient data 71 to 74. The correction coefficient data 75 corresponds to the reference tube first effective voltage value V1 and includes correction coefficients obtained for each channel of the detector assembly 108. Thereby, the first correction coefficient data 75 corresponding to the first effective voltage value V1 can be obtained. After the correction coefficient data 75 is obtained, the process proceeds to step ST14.

In step ST14, the processor 217 determines whether the view number v is v=n. Herein, v=1, and thus the process proceeds to step ST15.

In step ST15, the processor 217 increments the view number v. Herein, the view number v was v=1, and thus v=1 is incremented to v=2. After the view number v is incremented, the process returns to step ST12.

In step ST12, the processor 217 determines an effective voltage value for each view on the basis of the signal value including energy information of X-rays detected in the first sub-region 109B and the signal value including energy information of X-rays detected in the second sub-region 109A. Herein, v=2 is set, and thus an effective voltage value corresponding to the view v2 of the view number v=2 is obtained. View v2 is corresponds to low kV. In step ST12, the effective voltage value is obtained as follows.

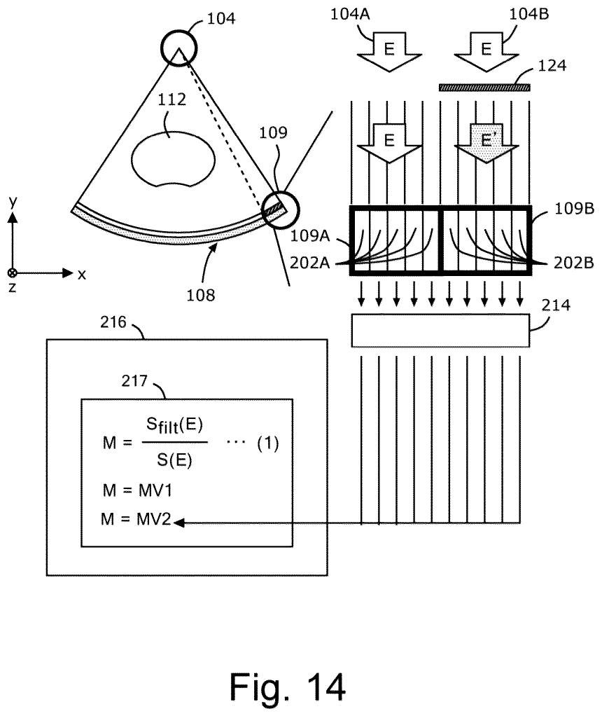

In step ST121, the processor 217 calculates a second index value M (=MV2) corresponding to the view v2 (low kV) by using equation (1) (see FIG. 14).

FIG. 14 is an explanatory diagram of a method for calculating the second index value M (=MV2) corresponding to the view v2 (low kV). The processor 217 determines the signal value Sfilt(E) and the signal value S(E) in equation (1) on the basis of data obtained from the reference region 109. The signal value Sfilt(E) is a third signal value including energy information of X-rays detected in the first sub-region 109B, and the signal value S(E) is a fourth signal value including energy information of X-rays detected in the second sub-region 109A. The processor substitutes the signal value Sfilt(E) and the signal value S(E) into equation (1) to calculate the second index value M (=MV2) corresponding to the view v2 (low kV). Once the second index value MV2 has been calculated, the process proceeds to step ST122.

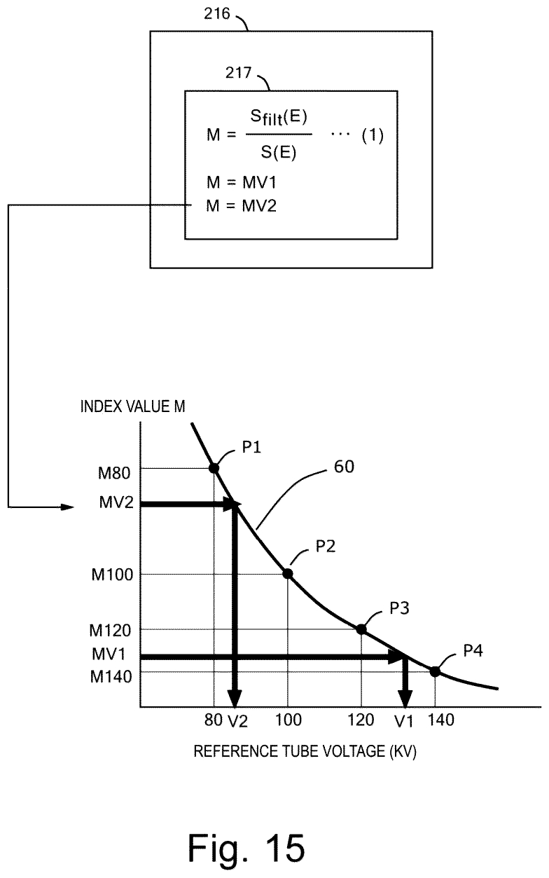

In step ST122, the processor 217 determines a second effective voltage value corresponding to the second index value MV2. FIG. 15 depicts the second effective voltage value V2 corresponding to the second index value MV2. The processor 217 identifies the second effective voltage value V2 corresponding to the second index value MV2 on the basis of the baseline 60. V2 is, for example, V2=85 kV. After the effective voltage value is determined, the process proceeds to step ST13.

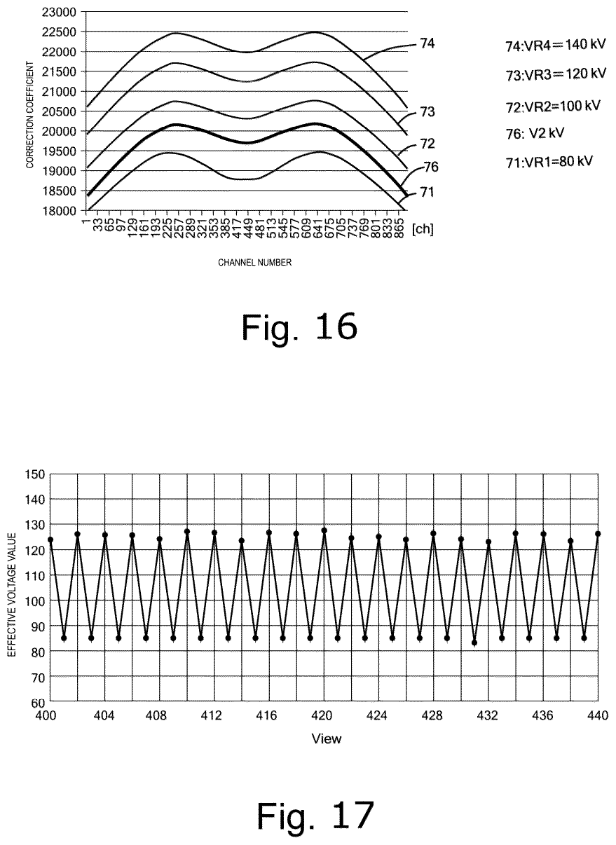

In step ST13, the processor calculates correction coefficient data corresponding to the low kV view v2 on the basis of the effective voltage value determined in step ST12 (see FIG. 16).

FIG. 16 is an explanatory diagram of a method for determining the correction coefficient data. The processor 217 interpolates correction coefficient data 76 corresponding to the second effective voltage value V2 on the basis of the second effective voltage value V2 and the plurality of reference correction coefficient data 71 to 74. The correction coefficient data 76 corresponds to the reference tube second effective voltage value V2 and includes correction coefficients obtained for each channel of the detector assembly 108. Thereby, the second correction coefficient data 76 corresponding to the second effective voltage value V2 can be obtained. After the correction coefficient data 76 is obtained, the process proceeds to step ST14.

In step ST14, the processor 217 determines whether or not correction coefficient data has been obtained for all views. Herein, the correction coefficient data 75 for view v1 (see FIG. 13) and correction coefficient data 76 for view v2 (see FIG. 16) have been calculated, but correction coefficient data for other views have not yet been calculated. Therefore, the process proceeds to step ST15.

In step ST15, the processor 217 increments the view number v. Herein, the view number v was v=2, and thus v=2 is incremented to v=3. After the view number v is incremented, the process returns to step ST12.

Similarly hereinafter, steps ST12 to ST15 are repeatedly executed for all views v1 to vn during one rotation of the X-ray tube 104 until correction coefficient data is obtained for each view. Furthermore, when the correction coefficient data for all the views v1 to vn corresponding to one rotation of the X-ray tube 104 have been obtained, the process proceeds to step ST16.

In step ST16, the processor 217 determines whether or not all correction coefficient data necessary for image reconstruction has been determined. For example, when the X-ray tube 104 moves to the second rotation and projection data for the second rotation is collected, correction coefficient data must be obtained for the projection data for the second rotation of the X-ray tube 104. Therefore, when the X-ray tube has made two rotations, the process proceeds to step ST11, where the view number is reset to v=1, and steps ST12 to ST15 are also executed for each view for the second rotation of the X-ray tube 104. Similarly hereinafter, steps ST11 to ST15 are executed every time the X-ray tube 104 makes one rotation. Furthermore, in step ST16, if the processor determines that the X-ray tube 104 has been able to determine all of the correction coefficient data necessary for image reconstruction, the process proceeds to step ST17.

In step ST17, the processor 217 reconstructs an image on the basis of the correction coefficient data obtained for each view. Thereby, the flow of FIG. 9 ends.

In the present embodiment, the detector assembly 108 of the CT system includes the filter 124 (see FIG. 5). The filter 124 is not disposed in the path of the X-rays 104A, but is disposed in the path of the X-rays 104B. The X-rays 104A are detected in the sub-region 109A (detector element 202A) without passing through the filter 124. On the other hand, the X-rays 104B pass through the filter 124, and X-rays 104C, the energy of which has been absorbed by the filter 124, are detected by the sub-region 109B (detector element 202B). Furthermore, an index value M is calculated using a signal value Sfilt(E) including energy information of X-rays that have passed through the filter 124 and a signal value S(E) including energy information of X-rays that have not passed through the filter 124. The index value M is used to obtain an effective voltage value.

In the present embodiment, as a preparation for obtaining the effective voltage value on the basis of the index value M, the baseline 60 (see FIG. 7), which defines the correspondence relationship between the index value M and the effective voltage value, is created. When creating the baseline 60, the four reference tube voltages VR1 to VR4 are set, and the four index values M80 to M140 corresponding to the four reference tube voltages VR1 to VR4 are calculated. Furthermore, the baseline 60 is created on the basis of the four reference tube voltages VR1 to VR4 and the four index values M80 to M140. The baseline 60 is stored in a storing device of the CT system (or in an external storing device). Each of the index values M80 to M140 is a value obtained by maintaining the tube voltage at a constant value of VR1 (=80 kV), VR2 (=100 kV), VR3 (=120 kV), and VR4 (=140 kV), respectively. Therefore, the index values M80 to M140 can be obtained without switching the tube voltage between the high kV and low kV, and thus each of the index values M80 to M140 can be calculated without being affected by the rotation speed of the gantry. Furthermore, the index values M80 to M140 are each a value calculated on the basis of the X-ray energy information. The energy of the X-rays basically depends on the tube voltage of the X-ray tube 104 and does not depend so much on the tube current, and therefore, the index values M80 to M140 can be calculated without being affected by the tube current. Therefore, each of the index values M80 to M140 is not substantially affected by the gantry rotation speed or tube current, and thus there is no need to obtain an index value for each combination of the gantry rotation speed and tube current, and one index value can be calculated for one reference tube voltage. In the present embodiment, the four index values M80 to M140 can be calculated for the four reference tube voltages VR1 to VR4 in this manner, and thus the baseline 60 representing the correspondence relationship between tube voltages and index values can be created without having to consider a large number of combinations of gantry rotation speeds and tube currents. Therefore, when imaging a subject using the kV switching technology, an effective voltage value can be identified for each view (each projection data) from the baseline 60 by calculating the index value M expressed by equation (1) on the basis of the X-rays detected in the reference region 109 during imaging of the subject. Therefore, correction coefficient data for each effective voltage value can be interpolated on the basis of the reference correction coefficient data 71 to 74 (see FIG. 8) stored in the storing device. As described above, an effective voltage value can be identified for each view (for each projection data), and thus correction coefficient data can be calculated for each view. As a result, a high quality image can be reconstructed. Furthermore, there is no need to obtain correction coefficient data for a large number of combinations of gantry rotation speeds and tube currents, and thus the work time required for calibration can be reduced. Furthermore, correction coefficient data can be obtained for each effective voltage value, and thus a scan can be performed with a combination of rotation speed and tube current desired by a user.

In order to specifically explain the effect of the present embodiment, FIG. 17 depicts an example of the relationship between the effective voltage value and the view when kV switching is executed.

FIG. 17 depicts effective voltage values corresponding to high kV and effective voltage values corresponding to low kV between views v400 and v440. Even numbered views represent effective voltage values corresponding to high kV, and odd numbered views represent effective voltage values corresponding to low kV. As can be seen from FIG. 17, the effective voltage value corresponding to high kV varies from view to view. Similarly, the effective voltage values corresponding to low kV also vary from view to view. Therefore, when obtaining a correction coefficient for each view using a conventional method, a combination of rotation speed and tube current must be prepared for each effective voltage value of each view as a preset and a calibration scan must be executed for each preset. However, the conventional method requires preparing a preset for each effective voltage value and executing a calibration scan, which results in an enormous amount of time being spent on calibration. Therefore, in the past, in order to prevent the calibration work time from increasing, the number of preset combinations of rotation speed and tube current that were prepared in advance was set to 16, for example, so that the time required for calibration would not be long. However, this poses a problem in that if the combination of rotation speed and tube current desired by a user is not included in the abovementioned 16 combinations, the subject cannot be scanned under the desired condition of the user.

Furthermore, when the number of views in a scan of one preset is 1,000, slight variations in the effective voltage value for each view occur, which creates the problem that if calibration data obtained for one preset is used for another preset, variations occur in the corrected data.

In contrast, in the present embodiment, if an index value M is calculated for each view, the effective voltage value corresponding to the index value M can be identified from the baseline 60, and thus a correction coefficient corresponding to the identified effective voltage value can be easily calculated by interpolation. Therefore, according to the present embodiment, the time required for calibration can be reduced, and the effect of being able to execute a scan of the subject can be executed with a combination of rotation speed and tube current desired by a user.

Note that in the present embodiment, an example is described in which the CT system 100 is used as a medical system. However, the present invention is not limited to the CT system 100 and can be applied to a system other than the CT system 100 (e.g., PET-CT system), so long as the medical system irradiates an X-ray source onto the subject 112.

1. A medical system, comprising:
an X-ray tube configured to be able to switch a tube voltage applied to the X-ray tube between a first tube voltage and a second tube voltage;
a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter; and
one or a plurality of processors for executing the following:
determining a first effective voltage value of the first tube voltage on the basis of energy information of X-rays detected in the first sub-region of the detector assembly and energy information of X-rays detected in the second sub-region of the detector assembly, when the first tube voltage is applied to the X-ray tube;
determining a second effective voltage value of the second tube voltage on the basis of energy information of X-rays detected in the first sub-region of the detector assembly and energy information of X-rays detected in the second sub-region of the detector assembly, when the second tube voltage is applied to the X-ray tube;
calculating a first correction coefficient required to correct for beam hardening on the basis of the first effective voltage value; and
calculating a second correction coefficient required to correct for beam hardening on the basis of the second effective voltage value.

2. The medical system according to claim 1, wherein determining the first effective voltage value includes:
determining the first effective voltage value on the basis of a first signal value including energy information of X-rays detected in the first sub-region and a second signal value including energy information of X-rays detected in the second sub-region, and
determining the second effective voltage value includes:
determining the second effective voltage value on the basis of a third signal value including energy information of X-rays detected in the first sub-region and a fourth signal value including energy information of X-rays detected in the second sub-region.

3. The medical system according to claim 2, wherein determining the first effective voltage value includes:
calculating a first index value on the basis of the first signal value and the second signal value; and
determining the first effective voltage value on the basis of the first index value, and determining the second effective voltage value includes:
calculating a second index value on the basis of the third signal value and the fourth signal value; and
determining the second effective voltage value on the basis of the second index value.

4. The medical system according to claim 3, wherein the first index value and the second index value are calculated by a ratio between a signal value including energy information of X-rays detected in the first sub-region and a signal value including energy information of X-rays detected in the second sub-region.

5. The medical system according to claim 1, wherein the one or more processors each calculate the first effective voltage value and the second effective voltage value on the basis of a baseline that associates a tube voltage with an index value.

6. The medical system according to claim 5, wherein the baseline is a line created on the basis of a plurality of reference tube voltages and a plurality of index values corresponding to the plurality of reference tube voltages, and
each of the plurality of index values is a ratio between a signal value including energy information of X-rays detected in the first sub-region and a signal value including energy information of X-rays detected in the second sub-region.

7. The medical system according to claim 6, wherein the one or plurality of processors executes the following:
obtaining first correction coefficient data corresponding to the first effective voltage value; and
obtaining second correction coefficient data corresponding to the second effective voltage value.

8. The medical system according to claim 7, wherein the first correction coefficient data and the second correction coefficient data include correction coefficients obtained for each channel of the detector assembly.

9. The medical system according to claim 7, wherein a plurality of reference correction coefficient data corresponding to the plurality of reference tube voltages are stored in a storing device in the medical system or in a storing device accessible by the medical system, and the one or plurality of processors executes the following:
calculating the first correction coefficient data on the basis of the first effective voltage value and the plurality of reference correction coefficient data; and
calculating the second correction coefficient data on the basis of the second effective voltage value and the plurality of reference correction coefficient data.

10. A non-transitory storing medium in which is stored an instruction executed by one or a plurality of processors of a medical system including:
an X-ray tube configured to be able to switch a tube voltage applied to the X-ray tube between a first tube voltage and a second tube voltage;
a detector assembly for detecting X-rays irradiated from the X-ray tube, the detector assembly including a reference region for detecting X-rays and a filter, the reference region including a first sub-region for detecting X-rays that have passed through the filter and a second sub-region for detecting X-rays that have not passed through the filter; and
the one or plurality of processors, wherein
the instruction, upon being executed by the one or plurality of processors, causes the one or plurality of processors to execute the following:
determining a first effective voltage value of the first tube voltage on the basis of energy information of X-rays detected in the first sub-region of the detector assembly and energy information of X-rays detected in the second sub-region of the detector assembly, when the first tube voltage is applied to the X-ray tube;
determining a second effective voltage value of the second tube voltage on the basis of energy information of X-rays detected in the first sub-region of the detector assembly and energy information of X-rays detected in the second sub-region of the detector assembly, when the second tube voltage is applied to the X-ray tube;
calculating a first correction coefficient required to correct for beam hardening on the basis of the first effective voltage value; and
calculating a second correction coefficient required to correct for beam hardening on the basis of the second effective voltage value.
